(19) United States
(12) Patent Application Publication
(10) Pub. No.: US 2010/0028261 A1
(43) Pub. Date: Feb. 4, 2010

(54) Molecular Specific Photoacoustic Imaging

(76) Inventors: Stanislav EMELIANOV, Austin, TX (US); Srivalleesha MALLIDI, Austin, TX (US); Konstantin SOKOLOV, Austin, TX (US); Timothy A. LARSON, Austin, TX (US); Bo WANG, Austin, TX (US); Evgeniya YANTSEN, Houston, TX (US)

Correspondence Address: Baker Botts L.L.P, 910 Louisiana Street, One Shell Plaza, HOUSTON, TX, 77002, US

(21) Appl. No.: 12/539,936

(22) Filed: Aug. 12, 2009

Related U.S. Application Data
(63) Continuation of application No. PCT/US08/53862, filed on Feb. 13, 2008.
(60) Provisional application No. 60/889,603, filed on Feb. 13, 2007.

(51) Int. Cl. A61K 49/00; G01N 33/567; G01N 33/574; C12Q 1/68

(52) U.S. Cl. 424/9.1; 435/7.21; 435/7.23; 435/6

(57) ABSTRACT

Methods relating to photoacoustic imaging of biological tissue are provided. One such method comprises contacting a biological tissue with a bioconjugate and irradiating the bioconjugate so as to generate an acoustic wave, wherein the bioconjugate comprises a nanoparticle and a moiety capable of selectively coupling a molecular marker. Suitable moieties include, among other things, epithelial growth factor receptor (EGFR).

### CROSS-REFERENCE TO RELATED APPLICATIONS

This application is a continuation of International Application No. PCT/US08/053862 filed Feb. 13, 2008, which claims the benefit of U.S. Provisional Application Ser. No. 60/889,603 filed Feb. 13, 2007, both of which are incorporated herein by reference.

### STATEMENT OF GOVERNMENT INTEREST

This disclosure was made with support under Grant Numbers EB008101, EB004963, CA110079 and CA103830, awarded by National Institutes of Health. The U.S. government has certain rights in the invention.

### BACKGROUND

There is a need for reliable, non-invasive imaging tools to detect, diagnose, and characterize cancer—one of the leading causes of death in the United States. The early detection of cancer is necessary for effective therapeutic outcome and is a primary indicator for long term survival. Moreover, demarcating tumor boundaries with high specificity is required to direct therapeutic interventions to tumor location and cause less or no damage to the surrounding healthy tissue.

Current imaging modalities suffer from many drawbacks. Optical imaging, for example, suffers from a shallow penetration depth on the order of millimeters. Additionally, ionizing imaging modalities, such as X-ray, CT, and PET, present safety concerns. Furthermore, current technologies employed in cancer treatments cause surrounding healthy tissue damage along with tumor necrosis.

Biological processes that lead to cancer occur at the molecular level. Nanotechnology offers unprecedented access to the machinery of living cells, and therefore provides the opportunity to study and interact with normal and cancerous cells in real time, at the molecular and cellular scales, and during the earliest stages of the cancer process. Studies have shown gold nanoparticles can be functionalized with antibodies to specifically bind to molecular markers that are indicative of highly proliferative cells or are overexpressed in different types of cancer.

Photoacoustic imaging is a technique that can provide functional information based on differences in optical absorption properties of the tissue constituents. The absorption of electromagnetic energy, such as light, and the subsequent emission of an acoustic wave by the tissue is the premise of photoacoustic imaging. Specifically for photoacoustic imaging, the tissue is irradiated with nanosecond pulses of low energy laser light. Broadband ultrasonic acoustic waves are generated within the irradiated volume, as the tissue absorbs the light and then undergoes rapid thermoelastic expansion. An ultrasonic sensor and associated receiver electronics are used to acquire the photoacoustic signal.

### DRAWINGS

Some specific example embodiments of the disclosure may be understood by referring, in part, to the following description and the accompanying drawings.

FIG. 1 shows a block diagram depicting an example embodiment of an imaging system for use with the present invention.

FIG. 2 shows the absorbance spectra of unlabeled, targeted, and non-targeted tissue samples normalized to the illumination lamp spectrum.

FIG. 3 shows darkfield, ultrasound, and photoacoustic images (λ=532 nm and 680 nm) of unlabeled, specifically targeted, and non-targeted tissue phantoms. The darkfield images show a 440 μm×340 μm field of view. The ultrasound and photoacoustic images show a 2 mm×1.67 mm field of view.

FIG. 4 shows a photograph of the injection sites on the mouse skin. The black solid line represents the imaging cross section. The red panel (dashed) represents the injection site of cells labeled with EGFR targeted gold nanoparticles. The green panel (solid) represents the injection site of cells mixed with gold nanoparticles with no molecular specificity.

FIG. 5 shows a block diagram of an exemplary combined photoacoustic and ultrasound imaging system.

FIG. 6 shows combined ultrasound and photoacoustic images of the mouse's abdominal region (a, b) before and (c, d) after injection of two gelatin solutions mixed with MDA-MB-468 (breast adenocarcinoma) cells labeled with EGFR targeted gold nanoparticles (red panel) hand cells mixed with polyethylene glycol-thiol (mPEG-SH) coated gold nanoparticles (green panel) respectively. The combined ultrasound and photoacoustic images measure 24 mm laterally by 16 mm axially.

FIG. 7 shows dark-field optical imaging of A) positive control, B) negative control, C) nonspecific blocking, and D) blocking with anti-EGFR antibody (C225) in a specificity assay to ensure molecular specificity of the anti-EGFR antibody.

FIG. 8 shows T2*-weighted images of normal mouse before (left) and after (right) injection of 100 uL, 10¹⁰ l/ml of iron/gold nanoparticles.

FIG. 9 shows side (left) and cross-sectional (right) views of a PVA phantom used in the vascular imaging example. Different compartments (right) are filled with a) gold nanoparticles suspended in 10% gelatin, b) 10% gelatin, c) macrophages loaded with gold nanoparticles, and d) macrophages in 10% gelatin.

FIG. 10 shows darkfield reflectance images of murine macrophages (left) and murine macrophages loaded with gold nanoparticles (right) using Xe illumination.

FIG. 11 shows the absorbance spectra of macrophages loaded with gold nanoparticles and gold nanoparticles only.

FIG. 12 shows an exemplary intravasular photoacoustic (IVPA) and intravascular ultrasound (IVUS) imaging system setup.

FIG. 13 shows IVUS (a,d), IVPA (b,e) and combined IVUS/IVPA (c,f) cross-sectional images of the vessel-mimicking phantom with four compartments (FIG. 9). The IVPA images were obtained at 532 nm (b) and 680 nm (e) wavelengths.

FIG. 14 shows IVPA images of the phantom at 690 nm, 710 nm, 730 nm and 750 nm optical wavelengths.

FIG. 15 shows the normalized photoacoustic signal strength from macrophages loaded with gold particles, 10% gelatin and 8% polyvinyl alcohol (PVA).

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to methods of imaging. More particularly, the present disclosure relates to photoacoustic microscopy methods for selectively imaging biological tissue.

One of the many advantages of the methods of the present disclosure is that photoacoustic imaging is a non-ionizing imaging method. Another advantage is that little or no additional equipment is needed for therapy. Similarly, the methods of the present disclosure provide for sequential monitoring of biological tissue during therapy.

In one embodiment, the present disclosure relates to a method comprising providing a bioconjugate, providing a biological tissue, contacting the biological tissue with the bioconjugate, irradiating the biological tissue to generate an acoustic wave, and detecting the acoustic wave. As used herein, the term “bioconjugate” is defined to include nanoparticles that have been functionalized with a biologically active moiety.

In certain embodiments, bioconjugates that may be used in conjunction with the methods of the present disclosure may comprise nanoparticles that are functionalized to specifically bind to a molecular marker. For example, highly proliferative or cancerous epithelial cells tend to overexpress epithelial growth factor receptor (“EGFR”). Thus, for embodiments wherein EGFR is the molecular marker of interest, one example of a bioconjugate that may be used in conjunction with the methods of the present disclosure includes gold nanoparticles that have been functionalized with anti-EGFR antibody. By way of explanation, and not of limitation, the anti-EGFR moiety of the bioconjugate may act as a targeting moiety and cause the bioconjugate particles to aggregate on the cellular membranes of cells that overexpress EGFR. This aggregation may lead to plasmon resonance coupling between nanoparticles and a red shift in the plasmon resonance frequency of the gold nanoparticle assembly. As used herein, the term “plasmonic nanoparticle” is defined to include any nanoparticle capable of exhibiting plasmon resonance coupling. The red-shift, among other things, may provide the opportunity to differentiate cancer cells from surrounding benign cells by using a combination of labeling with gold nanoparticles and multi-wavelength illumination.

Other suitable biologically active moieties include, but are not limited to, chlorotoxin, which specifically binds to neural gliomas, and the RGD peptide fragment (arginine-glycine-asparagine), which binds to integrins that are prevalent in tumor vasculature. Antibodies that target telomerase and matrix metalloproteinases may also be suitable moieties. The choice of a particular bioconjugate may depend, among other things, upon the tissue type to imaged, the target cell type, and the nanoparticle composition.

For bioconjugates comprising gold nanoparticles, the gold nanoparticles can be used as contrast agents in photoacoustic imaging, because of their strong optical absorption and scattering properties, and as therapeutic agents in photothermal therapy. As one of ordinary skill in the art is aware, the absorbance spectra of the gold nanoparticles can be modified by varying their shape and size. The gold nanoparticles can be tuned to resonate in the NIR region as light has higher penetration depth in the tissue at these wavelengths.

Biological tissues that are suitable for use with the methods of the present disclosure include any tissue that contains a selective receptor for a biologically active moiety of a bioconjugate. An example of such tissues includes, but is not limited to, epithelial tissue.

The imaging systems of the present disclosure generally comprise a light source and an ultrasonic sensor. Example light sources may include, but are not limited to, tunable pulsed lasers and fixed frequency pulsed lasers. Example ultrasonic sensors may include, but are not limited to, transducers. Examples of suitable transducers may include piezoelectric films, such as polyvinylidene fluoride, optical transducers, and optical interferometers. The imaging systems of the present disclosure may also comprise additional electronic and mechanical components such as a pulser/receiver, a digitizer, a motion controller, a three-dimensional positioning stage, and/or a delay switch. One of ordinary skill in the art, with the benefit of this disclosure, will recognize additional electronic and mechanical components that may be suitable for use in the methods of the present invention.

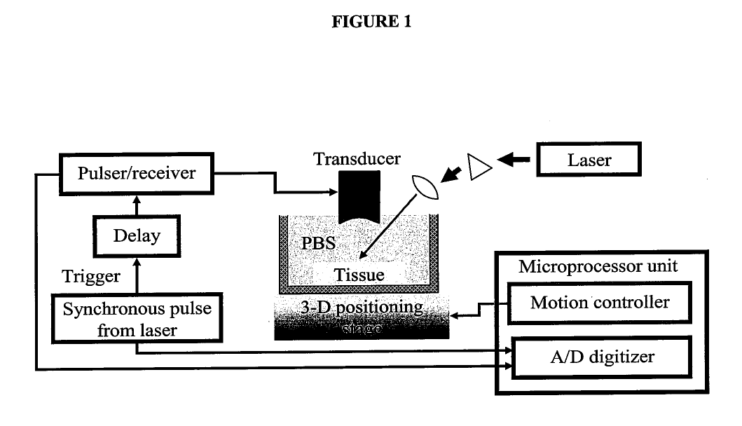

In certain embodiments, the ultrasonic sensor of an imaging system may also serve as a source of pulsed sound waves utilized to obtain an ultrasound image of the biological tissue. In such embodiments, a delay switch may be coupled to a synchronous trigger of a laser such that, after a photoacoustic image has been acquired, the acoustic detector will itself emit pulsed sound waves. The ultrasonic sensor may then detect echoes of these pulsed sound waves so that the echoes may be utilized to obtain an ultrasound image of the biological tissue. An example of an embodiment of this type imaging system is depicted in FIG. 1.

In certain embodiments, the aggregation of bioconjugates, for example gold nanoparticles conjugated with anti-EGFR antibodies, may be exploited to undertake molecule specific phototherapy. In such embodiments, the targeted bioconjugates may be used as guiding templates to create localized necrosis. This creation of localized necrosis, among other things, may result in little or no damage to healthy surrounding tissue. Specifically, localized necrosis may be caused by tissue ablation utilizing laser pulses of an energy higher than that required for photoacoustic imaging. Optionally, the progression of phototherapy may be monitored by ultrasound and/or photoacoustic imaging techniques as described herein.

In certain embodiments, the bioconjugates described herein may be used in other applications, including contrast agents for magnetic resonance imaging (MRI) or vascular imaging. Such embodiments may utilize the imaging systems described herein, with or without modifications for such applications that will be recognizable by one of ordinary skill in the art, with the benefit of this disclosure. In such embodiments, the bioconjugate composition may depend upon, among other things, the composition and location of the tissue and/or cell target to be imaged and the imaging system used.

In certain embodiments, the methods of the present disclosure may be used to monitor functional and morphological changes in tissue growth, including for example, in a variety of tissue engineering applications.

To facilitate a better understanding of the present disclosure, the following examples of certain aspects of some embodiments are given. In no way should the following examples be read to limit, or define, the entire scope of the invention.

### Examples

### Example 1

### Preparation of Tissue Phantoms

Specifically, three epithelial tissue phantoms consisting of human epithelial carcinoma cells (A431 keratinocyte) were used: (1) the control tissue sample with no gold nanoparticles; (2) the targeted tissue sample labeled with EGFR targeted gold nanoparticles; and (3) the non-targeted sample with nanoparticles coated with a polyethylene glycol-thiol (PEG-SH) layer which has no molecular specificity.

The 50 nm gold particles were synthesized via citrate reduction of HAuCl₄ under reflux. Anti-EGFR monoclonal antibody (clone 225) was purchased from Sigma and purified using a Centricon 100 kD MWCO filter. Antibodies were conjugated with gold nanoparticles using a protocol described in J. S. Aaron, J. OH, T. A. Larson, S. Kumar, T. E. Milner, and K. V. Sokolov, “Increased Optical Contrast in Imaging of Epidermal Growth fact Receptor Using Magnetically Actuated Hybrid Gold/Iron Oxide Nanoparticles,” Opt Express, (to be published), the relevant portions of which are herein incorporated by reference. Briefly, carbohydrate moieties on the antibodies' Fc region were oxidized to aldehyde groups via exposure to 100 mM NaIO₄ for 30 minutes and were allowed to covalently bind to a hydrazide portion of the bifunctional hydrazide-PEG-thiol linker (Sensopath Technologies, Inc.) to facilitate nanoparticle conjugation. This Ab-linker solution was diluted in HEPES pH 8 to 5 μg Ab/mL and mixed 1:1 with the colloid suspension (10¹² particles/mL) and allowed to conjugate via a thiol-gold binding reaction on a shaker at room temperature for 30 minutes. Subsequently, a small volume of PEG-SH (M.W. 2 kD, Shearwater) was added and allowed to react for another 30 minutes to passivate any remaining gold surface on the particles. To separate the conjugate from unbound antibody, the suspension was spun down at 1000 g for 30 minutes in the presence of 0.01% PEG polymer (M.W. 15 kD, Sigma) which was added as a surfactant to prevent aggregation during centrifugation. The pellet was resuspended in a 2% PEG (M.W. 15 kD, Sigma) in 1×PBS solution at the original particle concentration of 10¹² particles/mL. Particles for the non-targeted sample were conjugated only with PEG-SH and resuspended in a 2% PEG solution.

The A431 cells were purchased from American Type Culture Collection and cultured in DMEM supplemented with 5% fetal bovine serum (FBS) at 37° C. in a 5% CO₂ environment. Cells were harvested and resuspended in DMEM at a concentration of 2·10⁶ cells/mL and divided into three 450 μl aliquots. One of the aliquots was mixed with an equal volume of the anti-EGFR gold bioconjugate solution and allowed to interact for 45 minutes at room temperature. This sample was named the targeted sample. The other two aliquots were not exposed to the nanoparticles. The three cell suspensions were then spun down at 200 g and resuspended separately using 250 μL aliquots of a buffered collagen solution (2.1 mg/mL, pH 7.4). To determine the amount of nanoparticles attached to cells in the targeted sample, the optical density of the solution of gold bioconjugates at concentration used for labeling was compared to the optical density of the supernatant obtained after the labeled cells were spun down. The UV-Vis measurements showed ca. 260,000 particles per cell corresponding to approximately 25% of the total number of receptors per cell. The targeted sample contained approximately 4·10¹¹ gold nanoparticles/mL. Approximately 4·10¹² PEGylated Au particles/mL were added to the buffered collagen solution of the non-targeted sample. The control tissue sample had no gold nanoparticles. The cell/collagen solutions (200 μL) were pipetted into separate stacked spacers (0.5 mm silicone isolators, Molecular Probes) in Petri dishes for optical characterization and photoacoustic imaging. The cell/collagen solution in the Petri dish was allowed to gel in a 37° C. incubator for 1 hour. This procedure resulted in phantoms with randomly distributed cells in a three-dimensional collagen matrix. The 3-D arrangement of cells gives an opportunity to study imaging approaches having depth resolution. The phantoms were then covered with 50 μL of media and stored in an incubator for several hours prior to imaging.

### Example 2

### Optical Imaging of Tissue Phantoms

The tissue phantoms were characterized using a Leica DM 6000 upright microscope in epi-illuminated darkfield mode. A 75 W Xenon light source was used for illumination. Images were collected through a 20×, 0.5 NA darkfield objective and detected using a Q-Imaging Retiga EXi ultra-sensitive 12-bit CCD camera.

The extinction spectra were collected with a PARISS hyperspectral imaging device (Lightform, Inc.) in transmitted brightfield mode and a halogen light source. The hyperspectral device was coupled to the Leica microscope and was used to measure the extinction spectra at each pixel in the image. A single vertical section of the sample image was projected onto a prism through a 25 μm slit. The prism spectrally dispersed the one-dimensional image onto a two-dimensional Q-imaging Retiga EXi CCD detector. The sample was translocated laterally via a piezoelectric stage and the imaging process was repeated to construct the three-dimensional hyperspectral data cube. The spatial resolution of hyperspectral image was 1.25 μm and the spectral resolution was 1 nm. Transmitted brightfield spectral data cubes were acquired from 20×300 μm areas and normalized to the illumination lamp spectra, which was acquired through a blank slide containing only 1×PBS.

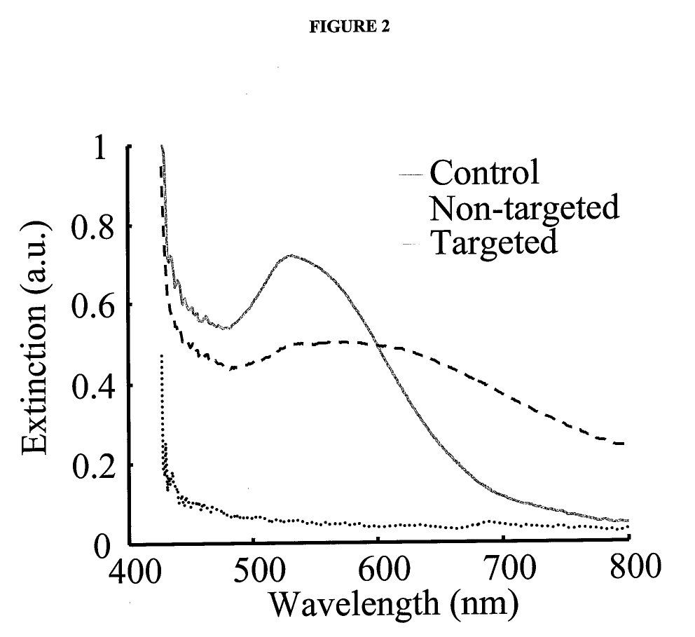

The extinction spectra of the control sample, the targeted sample, and the non-targeted sample are shown in FIG. 2. The control (blue dotted line) has low extinction in wavelength range of 450-800 μm. The non-targeted sample (green solid line) has an extinction peak at 520 nm, which is in excellent agreement with the extinction spectrum of a suspension of isolated gold nanoparticles. The targeted sample (red dashed line) has the peak red-shifted and broadened due to EGFR-mediated aggregation of gold nanoparticles. The extinction spectra were used as a guideline to gauge the difference in the optical properties of targeted and non-targeted tissue phantoms.

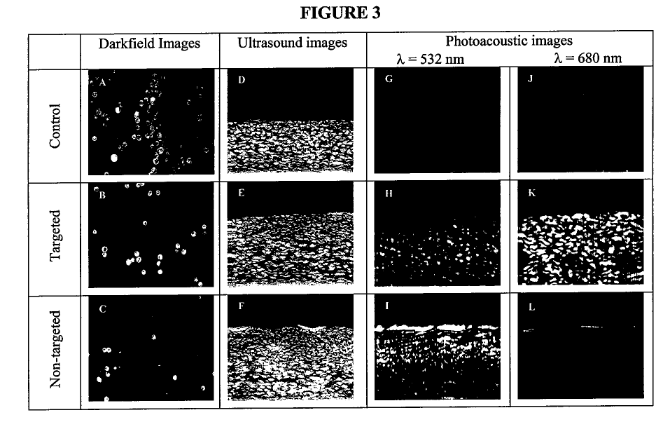

The darkfield images of the control, targeted and non-targeted phantoms are presented in FIGS. 3A, 3B and 3C. The control sample (FIG. 3A) does not contain any gold nanoparticles and hence the cells appear bluish white due to their intrinsic light scattering properties. The targeted sample (FIG. 3B) shows orange colored cells caused by the plasmon-resonance scattering of anti-EGFR conjugated gold nanoparticles which interact with EGFR molecules on the cytoplasmic membrane of A431 cells. The non-targeted tissue sample (FIG. 3C) has gold particles in suspension surrounding the cells. These isolated gold particles are associated with the greenish haze in the background surrounding the unlabeled A431 cells which appear bluish in the image.

### Example 3

### Ultrasonic and Photoacoustic Imaging of Tissue Phantoms

A block diagram of the experimental setup for ultrasound and photoacoustic imaging is shown in FIG. 1. A microprocessor unit with a custom built LabVIEW application controlled all modules of the imaging system including the ultrasound pulser/receiver, pulsed laser, data acquisition unit, and all motion axes needed for imaging via 3-D mechanical scanning. A 48 MHz single element focused ultrasound transducer (focal depth=5.5 mm, f#=1.4) was used to obtain both ultrasonic and photoacoustic images of the tissue samples. The tissue sample was attached to a 3-D positioning stage and placed in the focal region of the transducer. The Petri dish was filled with 1×PBS solution to maintain the appropriate pH in the medium surrounding the tissue phantoms.

A Q-switched Nd:YAG laser operating at wavelength of 532 nm (5 ns pulses, 20 Hz pulse repetition frequency) was used to obtain photoacoustic images of the samples. The tissue phantoms were also imaged with a tunable OPO laser operating at a wavelength of 680 nm and capable of producing 7 ns pulses at 10 Hz pulse repetition frequency. The ultrasound and photoacoustic images were obtained by mechanically scanning the tissue samples over the desired region. The sampling interval of the mechanical scan (12 μm) was set to be smaller than half the beamwidth of the ultrasonic transducer (42 μm) to satisfy the Nyquist criterion. The photoacoustic response of the sample being imaged was captured using the same receiver electronics as ultrasonic imaging. Specifically the master trigger from the laser source, delayed by several microseconds, was sent to the pulser/receiver to initiate the pulse-echo ultrasound regime. The synchronous trigger from the laser also commenced the data acquisition with 8-bit, 500 MHz digitizer and the data was stored for offline processing. In addition, digital bandpass (20-70 MHz) filtering was employed to reduce noise in the signals. An acquired A-line, therefore, contained the spatially co-registered photoacoustic signal followed by the conventional ultrasound signal.

The ultrasonic images of the three tissue phantoms are presented in of FIGS. 3D, 3E and 3F. The images do not reveal any information regarding the isolated or clustered state of the gold nanoparticles due to insufficient acoustic contrast. The photoacoustic images of the tissue samples shown in FIGS. 3G, 3H and 3I were obtained with 532 nm laser irradiation and images presented in FIGS. 3J, 3K and 3L were obtained using 680 nm laser irradiation. All photoacoustic images are displayed using the same dynamic range. The photoacoustic images of the control sample do not show any signals at both 532 nm (FIG. 3G) and 680 nm (FIG. 3J), indicating that the tissue absorbs less light at these wavelengths as compared to the other two tissue samples. The faint signal in the lower region of the photoacoustic images (FIGS. 3G and 3J) was due to absorption of the laser by the plastic bottom of the Petri dish that is holding the tissue sample.

At 532 nm laser irradiation, the photoacoustic image of the non-targeted sample (FIG. 3I) indicates higher optical absorbance than the targeted sample (FIG. 3H). Due to the high extinction coefficient of the non-targeted sample at 532 nm, the laser fluence decreases exponentially with depth. Depth dependent compensation was applied to photoacoustic signals in FIG. 3I to compensate for the signal loss due to decrease in the laser light fluence. At 680 nm illumination, very little photoacoustic response was obtained in the non-targeted sample (FIG. 3L), unlike the targeted sample that produced signal from the entire tissue phantom slab of 1 mm thickness (FIG. 3K). Hence in congruence with the hyperspectral analysis (FIG. 2), a relatively low overall extinction coefficient was observed in the non-targeted sample as compared to targeted sample at 680 nm. Thus, specific targeting of gold nanoparticles to EGFR molecules that are overexpressed in certain types of cancer results in significant increase in the photoacoustic signal in the red optical region. The increase in photoacoustic signal may be due to EGFR mediated assembly of gold nanoparticles on the cytoplasmic membrane of the cancerous cells; this leads to plasmon resonance coupling between adjacent gold particles and changes in their extinction spectra which are shown in FIG. 2.

### Example 4

### In Vivo Imaging

Bioconjugation of gold nanoparticles and preparation of gelatin solution with MDA-MB-468 cells

50 nm gold particles were synthesized via citrate reduction of HAuCl4 under reflux. Anti-EGFR monoclonal antibody (clone 225) was purchased from Sigma (Sigma-Aldrich Inc., Saint Louis, Mo.) and purified using a Centricon 100 kD MWCO filter. The carbohydrate moieties on the antibodies' Fc region were oxidized to aldehyde groups via exposure to 100 mM NaIO4 for 30 minutes and were allowed to covalently bind to a hydrazide portion of the bifunctional hydrazide-PEG-thiol linker (Sensopath Technologies, Inc.) to facilitate nanoparticle conjugation. Diluent of Ab-linker was exchanged to HEPES pH 8 to the final concentration of antibody 50 μg Ab/mL. This solution was mixed 1:10 with the gold colloid suspension (4×10¹⁰ particles/mL) and allowed to conjugate via a thiol-gold binding reaction on a shaker at room temperature for 30 minutes. Subsequently, a small volume of PEG-SH (M.W. 2 kD, Shearwater) was added and allowed to react for another 30 minutes to passivate any remaining gold surface on the particles. To separate the conjugate from unbound antibody, the suspension was centrifuged at 1000×g for 30 minutes in the presence of 0.01% PEG polymer (M.W. 15 kD, Sigma) which was added as a surfactant to prevent aggregation during centrifugation. The pellet was resuspended in a phenol red free DMEM at the concentration of 4·10¹¹ particles/mL. Particles for the non-targeted injection were conjugated only with PEG-SH and centrifuged.

The MDA-MB-468 cells were cultured in MEM supplemented with 10% fetal bovine serum at 37° C. in a 5% CO₂ environment. To specifically label the cells with gold nanoparticles, the cells were harvested and resuspended in anti-EGFR gold conjugate solution at a concentration of 2·10⁷ cells/mL and incubated for one hour at 37° C. Brief estimation of number of gold conjugates bound to cells gave ˜200,000 conjugates/cell. The cells harvested and resuspended in phenol red free DMEM were used for the second gelatin solution. Both cells aliquots were centrifuged and the supernatant was removed. Cells labeled with targeted gold nanoparticles were resuspended in warm (˜37° C.) gelatin solution (10% by weight) at a concentration of 9·10⁶ cells/mL. The second aliquot of was resuspended in gelatin solution (10% by weight) and PEGylated gold nanoparticles to obtain the final concentration of approximately 10¹² gold nanoparticles/mL. Both the gelatin suspensions were maintained at approximately 37° C. and were injected into the mouse abdomen using 30-gauge needle syringe.

Intraperitoneal Injection of Gelatin Solution in Mouse

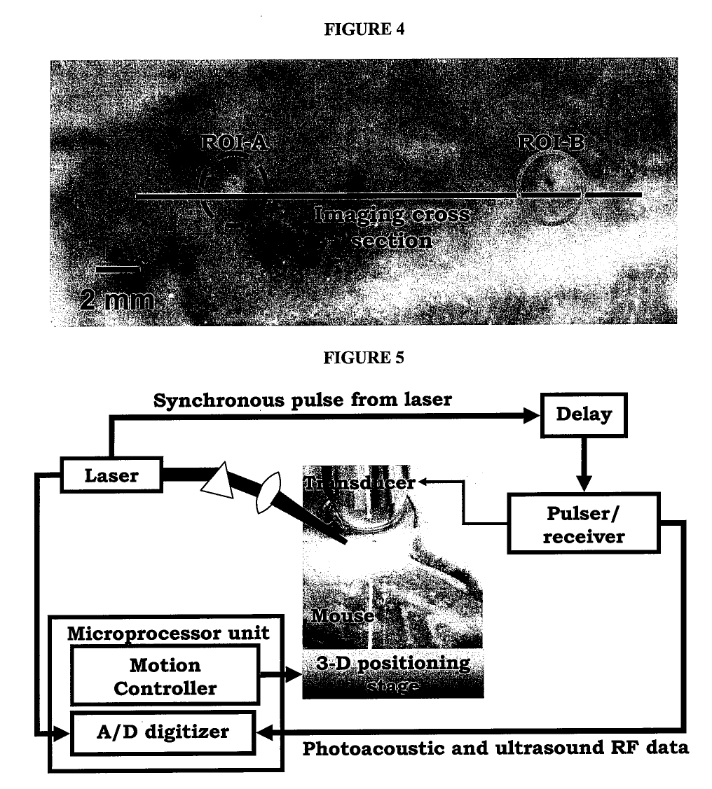

An euthanized BL6 mouse was obtained from the Animal Resource Center at The University of Texas at Austin. A commercially available depilatory solution was used to remove hair from the abdominal region of the mouse. To mimic a tumor specifically targeted with gold nanoparticles, 500 μL gelatin solution with MDA-MB-468 (breast adenocarcinoma) cells labeled with EGFR targeted gold nanoparticles was injected into the abdominal cavity of the mouse (FIG. 4, ROI-A). A second injection of 200 μL gelatin solution mixed with MDA-MB-468 cells and nanoparticles coated only with a polyethylene glycol-thiol (mPEG-SH) layer and having no molecular specificity was injected (FIG. 4, ROI-B) approximately 15 mm away from the ROI-A injection site. The colder environment of the mouse body facilitated the hardening of the gelatin solution inside the abdominal cavity. The tumor mimicking gelatin clumps in the abdominal cavity of the mouse were approximately 6-7 mm deep. The photoacoustic and ultrasound images from the same cross section of the abdomen region (FIG. 4) were obtained before and after the injection, and compared.

Experimental Setup for Combined Photoacoustic and Ultrasound Imaging

A block diagram of the experimental setup for the combined photoacoustic and ultrasound imaging is shown in FIG. 5. The imaging system consists of a microprocessor unit with a custom built LabVIEW application that controls the ultrasound pulser/receiver, pulsed lasers, data acquisition unit, and all motion axes needed for 3-D mechanical scanning. A 25 MHz single element focused ultrasound transducer (focal depth=25.4 mm, f#=4) was used to obtain both ultrasonic and photoacoustic images of the tissue phantoms. Either a Q-switched Nd:YAG laser (532 nm wavelength, 5 ns pulses, 20 Hz pulse repetition frequency) or a tunable OPO laser system (680 nm wavelength, 7 ns pulses, 10 Hz pulse repetition frequency) was used to generate photoacoustic transients.

The mouse was placed in a water tank attached to a 3-D positioning stage. The 2-D photoacoustic and ultrasound images were obtained by mechanically scanning over the desired region with 100 μm lateral steps to satisfy Nyquist criterion. At each step, the pulsed laser light irradiated the sample and the trigger signal from the laser source initiated the data acquisition by an 8-bit, 500 MHz digitizer. The same trigger signal, delayed by several microseconds, was sent to the pulser/receiver to initiate the pulse-echo ultrasound imaging. Therefore, a captured A-line contained the photoacoustic signal and the conventional ultrasound radio-frequency (RF) data separated by the user defined delay. The A-line records obtained at each lateral step of the mechanical scan were processed offline to obtain spatially co-registered 2-D photoacoustic and ultrasound images. During the offline processing, the photoacoustic and ultrasound signals are extracted from the A-line records and a digital bandpass (5-45 MHz) filter was applied to these raw RF signals to reduce noise. The analytic signals obtained from the photoacoustic and ultrasound RF data were spatially interpolated. The photoacoustic image was overlaid on the corresponding ultrasound image in the region of interest and displayed over a 40 dB dynamic range.

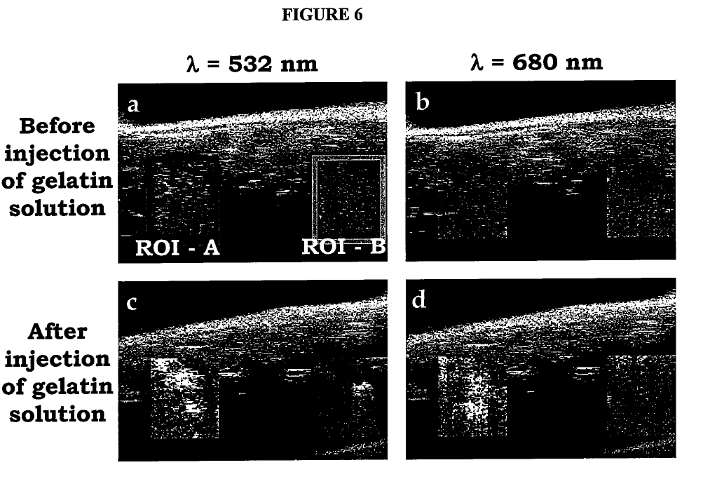

The combined ultrasound and photoacoustic images of the mouse abdomen before (FIGS. 6a and 6b) and after (FIGS. 6c and 6d) the intraperitoneal injection of two gelatin solutions mixed with MDA-MB-468 cells labeled with EGFR targeted gold nanoparticles (ROI-A, red panel) and cells mixed with mPEG-SH coated gold nanoparticles (ROI-B, green panel) are presented in FIG. 6. The images measure 24 mm laterally and 16 mm axially. The images clearly depict injection sites located 6-7 mm deep in the abdominal cavity of the mouse as it is evident from the significant increase of the photoacoustic signal magnitude (FIGS. 6c and 6d) in the region of interest.

In congruence with the absorbance spectra shown in FIG. 2, the cells mixed with PEGylated gold nanoparticles produce greater photoacoustic signal with 532 nm laser irradiation (FIG. 6c) in ROI-B. It can also be observed the photoacoustic signal in region B (FIG. 6d) is similar to the photoacoustic signal obtained before injection (FIG. 6b) with 680 nm laser irradiation. The ROI-A, where the cells labeled with EGFR targeted gold nanoparticles were injected, showed significant increase in photoacoustic signal both at 532 nm and 680 nm wavelength laser illuminations. When targeted gold nanoparticles bind to EGFR they tend to cluster in the same spatial distribution as EGF receptors, which are known to form closely spaced assemblies upon activation with EGF followed by endocytoses of the receptors. The receptor-mediated aggregation of gold nanoparticles causes plasmon coupling of the clustered nanoparticles, leading to an optical red-shift of the plasmon resonances and an increase in absorption in the red region (FIG. 2). Thus, specific targeting of gold nanoparticles to EGFR molecules that are overexpressed in certain types of cancer results in significant increase in the photoacoustic signal in the red optical region. A decrease in the photoacoustic signal from ROI-A at wavelengths greater than 680 nm has also been observed.

While the photoacoustic images shown in FIG. 6 were not obtained in real-time, such real-time imaging is contemplated by the present disclosure. The mechanical scanning of the single element transducer and the pulse repetition rate of the laser increases the time needed to acquire the combined ultrasound and photoacoustic images. Real-time photoacoustic imaging may be accomplished using array transducers operating in 5-10 MHz frequency range. Moreover, the minimum concentration of specifically targeted gold nanoparticles required to obtain sufficient contrast in photoacoustic images from the deeply embedded tumors has to be determined.

### Example 5

### Specificity Assay

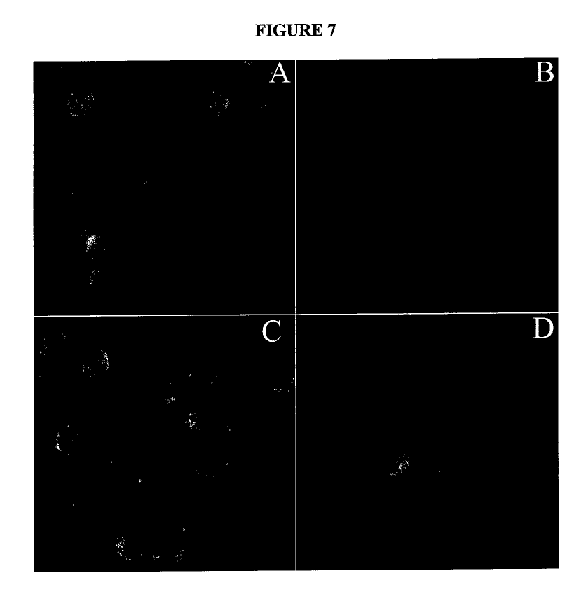

To ensure molecular specificity, A431 cells were exposed to excess anti-EGFR antibody (C225) in PBS to block available receptors. A separate aliquot of A431 cells was exposed to non-specific (anti-goat) antibody to verify that the blocking was molecular specific. A431 cells not exposed to antibody were used as the positive control. Finally, MDA-MB-435 cells, which do not express EGFR, were used as the negative control. Targeted anti-EGFR nanoparticles were added to the two blocked samples and the positive and negative controls and allowed to interact for 20 minutes. The suspensions were then centrifuged, the O.D. of the supernatants were collected and compared with the original nanoparticle solution (diluted appropriately in PBS) to determine labeling efficiency, and the cells were imaged in dark-field reflectance mode to verify the results (FIG. 7). Blocking cells with an excess of C225 antibody resulted in a 26× decrease in labeling efficiency as compared to the positive control, while cells exposed to nonspecific IgG showed no decrease in labeling efficiency. MDA-MB-435 cells, which do not express EGFR, did not show any particle uptake.

### Example 6

### Use as MRI Contrast Agent

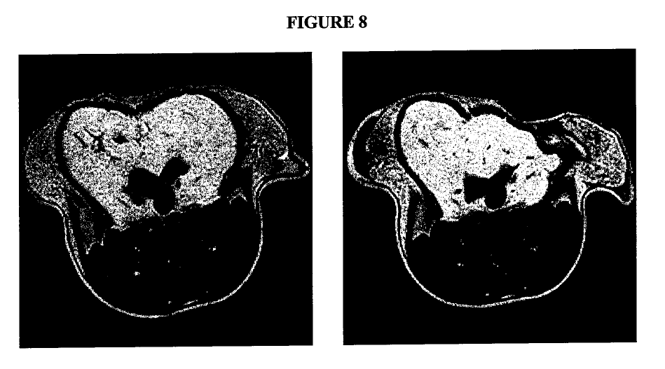

Ten nanometer diameter iron oxide nanoparticles were synthesized via reduction of FeCl₂ and FeCl₃ in a 2:1 molar ratio. Gold ions were reduced onto the surface of the iron via an iterative hydroxylamine seeding technique resulting in ca. 50 nm diameter particles. Following synthesis the particles were functionalized with anti-EGFR Ab (Neomarker c225). The nanoparticles were injected into a mouse to demonstrate in vivo MR contrast. T1-, T2-, and T2*-weighted images were collected before and after injection of 100 uL, 10¹⁰ particles/ml into the abdominal fat pad of a normal mouse. Imaging was done using a 4.7 T Biospec experimental MR system (Bruker Biospin MRI, Billerica, Mass., USA). The functionalized nanoparticles were clearly distinguished in a mouse in vivo, providing negative T2 and T2* contrast. Representative T2-weighted images are shown in FIG. 8.

### Example 7

### Use in Vascular Imaging

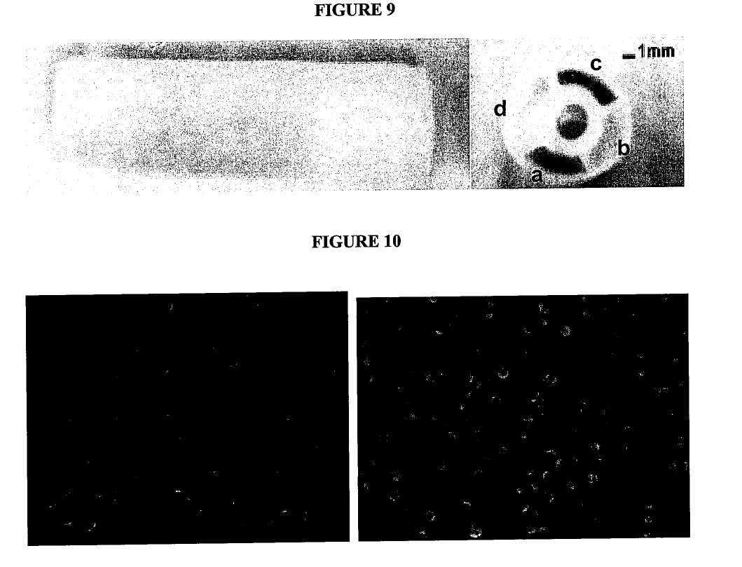

Vascular imaging experiments were performed using tissue-mimicking phantoms simulating a vessel wall with occlusions (FIG. 9). The vessel wall was made of 8% polyvinyl alcohol (PVA)—a polymer with tissue-like optical scattering properties. To provide acoustic scattering, 0.4% silica by weight was added to the background material. The phantom, subjected to three freeze/thaw cycles, was about 25 mm in length and 6 mm in diameter. Within the phantom, four compartments near and around the lumen were made. Each of the four compartments was filled with 10% gelatin gel containing a) gold nanoparticles, b) gelatin only, c) murine macrophages loaded with gold nanoparticles, and d) murine macrophages without nanoparticles.

First, 50 nm diameter spherical gold nanoparticles were synthesized via citrate reduction of HAuCl₄ under reflux. Then, they were coated with polyethylene glycol-thiol (PEG-SH) to passivate the surface of the nanoparticles. A small volume of 10⁻⁴M mPEG-SH solution (MW 5000 kD, Shearwater) was added to the particle suspension and allowed to react for 30 minutes. After incubation, small volume of 2% PEG polymer (MW 15 kD, Sigma) was added to the mixture to serve as surfactant and prevent aggregation of nanoparticles during centrifugation. The mixture was then centrifuged at 2500×g for 30 minutes, resulting in the pellet of PEGylated gold nanoparticles. Finally, the pellet was resuspended in either warm 10% gelatin (35-40° C., temperature of gelatinization 24° C.) with approximate concentration of 2×10¹¹ particles/ml or phenol red free DMEM.

The mouse monocytes—macrophages (J774A.1 cell line) are characterized by a high rate of non-specific uptake, similar to most cells of macrophage phenotype. Cells were cultured in DMEM supplemented with 5% FBS at 37° C. in 5% CO₂. To load cells with gold nanoparticles (FIG. 10), the cells were incubated with the suspension of PEGylated nanoparticles (approximate concentration—10¹⁰ particles/ml) in phenol red free DMEM overnight. To determine the number of nanoparticles internalized by the cells, the optical density of the incubation medium was measured at the absorbance peak of a 50 nm gold nanoparticle suspension before and after incubation with cells. The total quantity of nanoparticles inside the cells was then divided by the number of cells. This quantity varied in the range of 5×10³-6×10⁴ nanoparticles per cell. Starting from this number, the number of cells needed to get concentration of nanoparticles equal to those in the gel with the nanoparticles only was determined, i.e., 2×10¹¹ particles/ml. The gold nanoparticles endocytosed by the macrophages are located in intracellular vehicles in an aggregated state.

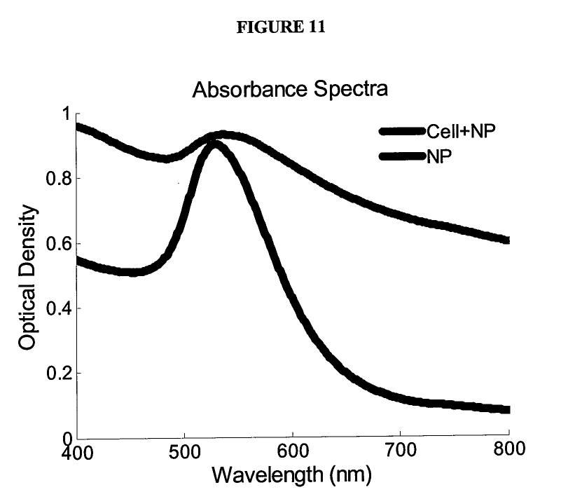

The optical absorbance spectrum of pure gold nanoparticles has a peak at 530 nm, whereas the spectrum of macrophages loaded with gold particles has a peak in the region of 540 nm, i.e. there is a slight red shift of the spectrum compared to pure nanoparticles (FIG. 11). More importantly, however, is that the absorption peak is broader—starting at 530 nm wavelength and higher, the absorbance spectra of cells loaded with gold nanoparticles is higher than that of pure nanoparticles.

Prior to the imaging experiments, the intact and loaded cells were harvested, mixed with warm (35° C.) 10% gelatin and loaded into the corresponding compartments of the phantom (FIG. 9). Concentration of the normal cells in gelatin was equal to that of cells loaded with nanoparticles. After placing loaded macrophages, normal (control) macrophages, gold nanoparticles and gelatin into the corresponding compartments (FIG. 9), the phantom was preserved in PBS for imaging.

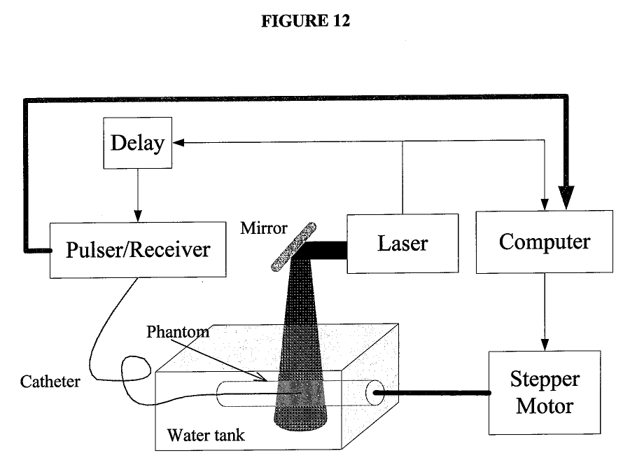

During the imaging experiment, the phantom was placed in a water tank filled with a physiological solution (FIG. 12). A 40 MHz IVUS imaging catheter (Boston Scientific, Inc.) was placed in the center of the cylindrical lumen of the phantom. The phantom was irradiated from the top using either an Nd:YAG pulsed laser (532 nm wavelength) or a tunable OPO pulsed laser system (680-950 nm wavelength). Immediately after the laser pulse, photoacoustic A-line signal was recorded using an IVUS transducer. After an 8 μs delay, ultrasound pulse was generated and ultrasound pulse-echo signal was received using the same transducer. The RF data was captured using 14-bit, 200 MHz A/D digitizer (Gage Applied, Inc.). The phantom was then rotated around the longitudinal axis using a stepper motor where at each angular position both IVPA and IVUS A-line were collected. As the phantom for rotated 360°, two co-registered IVUS and IVPA images of the phantom's cross-section were collected.

Finally, the spectroscopic IVPA imaging was performed by 10 nm incremental change of optical wavelength from 680 nm to 750 nm. At each wavelength, the photoacoustic transients were detected and the energy of the photoacoustic response (integral or area under the curve) within small region of interest was computed. The wavelength-dependent behavior of the IVPA signal was then analyzed to reveal spectral properties of optical absorption within specific regions of the phantom.

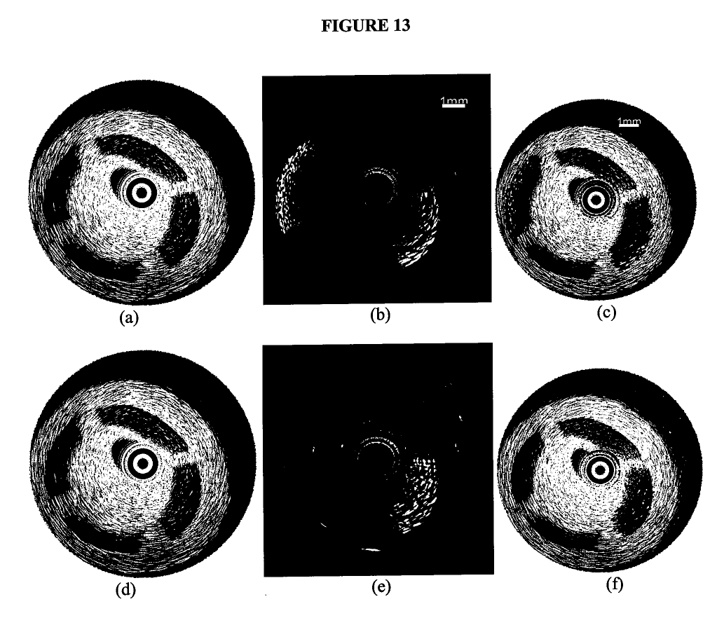

The results of the IVUS and IVPA imaging studies at two discrete optical wavelengths—532 nm and 680 nm, are presented in FIG. 13. In IVUS images (FIGS. 13(a) and 13(d)), the phantom geometry (lumen, wall thickness, etc.) and all four compartments within the vessel wall of the phantom can be easily identified. Two compartments with macrophages can be distinguished from the remaining two compartments containing gelatin or gold nanoparticles suspended in gelatin. Indeed, the compartments filled with macrophages are characterized by a presence of weak echo signal (small intensity of grayscale ultrasound) while the other compartments produces no ultrasound signal. Clearly, neither gelatin nor gold nanoparticles affect contrast in IVUS images—gold nanoparticles cannot be seen from IVUS images.

In contrast with IVUS images, two compartments with gold nanoparticles are visualized in the IVPA image obtained at 532 nm wavelength (FIG. 13(b))—there is a strong photoacoustic signal measured from these compartments and no measurable photoacoustic signal was detected from other compartments containing macrophages and/or gelatin. Because of the attenuation of light as it travels through the phantom, gold nanoparticles located closer to the lumen (and, therefore, further away from the laser source) generate a lower photoacoustic signal. Also, due to increased scattering of aggregated nanoparticles and the scattering of the cells, laser light fluence is distributed more homogeneously inside the compartment with loaded macrophages, thus, the photoacoustic speckle spot size in this compartment is bigger. However, only limited structural information is available from the IVPA image. By combining spatially co-registered IVUS and IVPA images, it is possible to identify the localization of compartments with gold nanoparticle within the overall structure of the vessel (FIG. 13(c)).

Therefore, macrophages loaded with gold nanoparticles can be easily identified in IVPA or combined IVUS/IVPA images (FIG. 5(b)-(c)). It is anticipated that the photoacoustic signal from gold nanostructures will be significantly higher than the signal measured from macrophages, fibrotic tissue, lipid deposits, etc. However, luminal blood is a very strong optical absorber at this wavelength. In addition, gold nanoparticles within the blood and near the vessel wall will also produce undesired photoacoustic response.

To avoid the effect of strong optical absorption of blood, the imaging wavelength in IVPA imaging can be changed. Indeed, luminal blood has minimum optical absorption at 680 nm wavelength. In addition, there is a sharp decrease of optical absorption in non-aggregated (i.e., single) nanoparticles after 530 nm and specifically at 680 nm while the absorption spectra of aggregated nanoparticle is relatively broad (FIG. 11). Therefore, the photoacoustic signal from nanoparticles passively attached to the vessel wall or within the blood stream (i.e., nanoparticles suspended in gelatin in our experimental studies) should be much smaller at 680 nm wavelength compared to aggregated nanoparticles (i.e., macrophages loaded with gold nanoparticles).

The IVPA imaging studies performed at 680 nm wavelength (FIG. 13(d)-(e)) confirm that the compartment containing macrophages with aggregated particles produces strong photoacoustic signal while the photoacoustic signal from gold nanoparticles suspended in gelatin is drastically reduced (FIG. 13(e)). This phenomenon can potentially be used to visualize molecularly targeted components in the artery thus allowing the assessment of both plaque morphology and composition. Overall, the results presented in FIG. 13 demonstrate cell specific intravascular photoacoustic imaging using gold nanoparticles. A variety of metal geometries including nanospheres, nanowires nanoshells, nanocages and nanocrescents can be used in photoacoustic imaging.

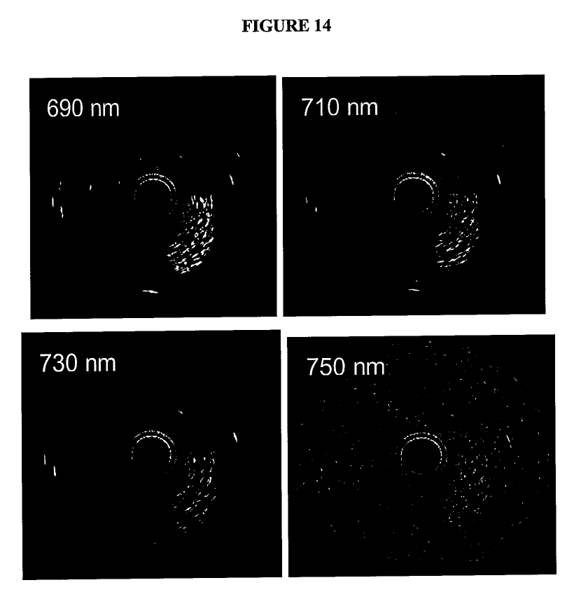

To further confirm the presence of aggregated particles and potentially to differentiate the gold nanoparticles from other tissue constituents such as blood or lipid, spectroscopic IVPA imaging can be performed. FIG. 14 demonstrates IVPA images acquired at several optical wavelengths: 690 nm, 710 nm, 730 nm and 750 nm. As expected (FIG. 11), the photo-acoustic response from the compartment with loaded macrophages is gradually decreasing. At 750 nm wavelength excitation, the IVPA response from loaded macrophages is almost the same as from other regions within the phantom.

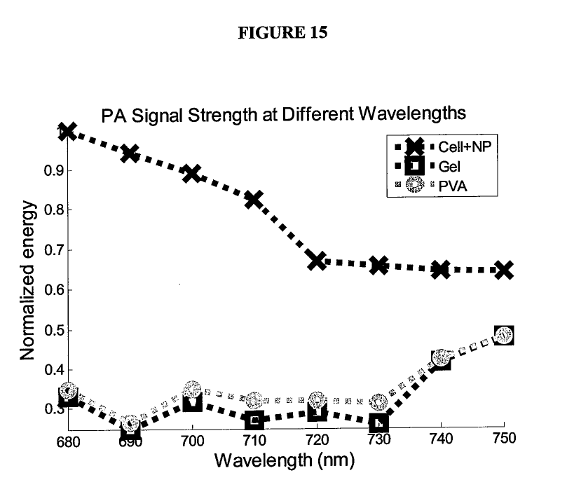

FIG. 15 shows quantitative behavior of wavelength-dependent photoacoustic response from several specific parts of the phantom: macrophages loaded with gold nanoparticles, 10% gelatin and PVA. As expected, the largest normalized energy of IVPA signals was detected from loaded macrophages at 680 nm wavelength. As the wavelength increases, the IVPA signal amplitude from loaded macrophages decreases. This measurement correlates qualitatively with the direct measurements of optical spectrum of aggregated nanoparticles (FIG. 11). In opposite, the photoacoustic response of gelatin and PVA polymer increases in this optical range. Such trend of increased photoacoustic signal with increased wavelength is indicative for many soft tissues including blood, muscle and fat. Therefore, since most components in the artery have increased absorption spectra at wavelength from 680 nm to 750 nm, and aggregated nanoparticles exhibit an opposite behavior, gold nanoparticles may be distinguished using spectroscopic IVPA imaging.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A method comprising: contacting a biological tissue with a bioconjugate and irradiating the bioconjugate so as to generate an acoustic wave, wherein the bioconjugate comprises a nanoparticle and a moiety capable of selectively coupling a molecular marker.

2. The method of claim 1 further comprising detecting the acoustic wave and utilizing the detected acoustic wave to generate an image of the biological tissue.

3. The method of claim 2 wherein the biological tissue comprises cancer cells.

4. The method of claim 2 wherein the biological tissue is a blood vessel.

5. The method of claim 1 wherein the bioconjugate comprises plasmonic nanoparticles.

6. The method of claim 1, wherein the moiety is covalently attached to gold nanoparticles.

7. The method of claim 1, wherein the bioconjugate further comprises thiolated polyethylene glycol covalently attached to gold nanoparticles.

8. The method of claim 1, wherein the moiety is selected from the group consisting of an antibody, an antibody fragment, a peptide fragment, a DNA fragment, and an RNA fragment.

9. The method of claim 1 further comprising providing a transducer, wherein the transducer is utilized to detect the acoustic wave.

10. The method of claim 1, wherein the biological tissue comprises the molecular marker and wherein coupling of the moiety to the molecular marker can cause aggregation of the bioconjugate.

11. The method of claim 10, further comprising analyzing a detected acoustic wave to indicate a distribution of the molecular marker.

12. The method of claim 1 wherein the bioconjugate has a peak absorption at a first frequency when nonaggregated and a peak absorption at a second frequency when aggregated.

13. The method of claim 12 further comprising irradiating the bioconjugate at the first frequency so as to generate a first acoustic wave and irradiating the bioconjugate at the second frequency so as to generate a second acoustic wave.

14. The method of claim 13, wherein the step of irradiating the bioconjugate at the first frequency is not simultaneous to the step of irradiating the bioconjugate at the second frequency.

15. The method of claim 13, further comprising detecting the first acoustic wave and detecting the second acoustic wave.

16. The method of claim 15 wherein the step of detecting the first acoustic wave is not simultaneous to the step of detecting the second acoustic wave.

17. The method of claim 1 further comprising detecting the acoustic wave with a transducer; generating a pulsed acoustic wave; detecting an echo of the pulsed acoustic wave with the transducer; and generating an image of the biological tissue.

18. The method of claim 1 further comprising detecting the acoustic wave; utilizing the detected acoustic wave to generate an image of the biological tissue; and wherein the step of irradiating the bioconjugate is implemented so as to generate the acoustic wave.

19. The method of claim 1 further comprising ablating the biological tissue.

20. A method comprising: administering a bioconjugate to an organism having biological tissue, wherein the bioconjugate comprises a moiety capable of selectively coupling a molecular marker; and irradiating the bioconjugate so as to generate an acoustic wave.

21. The method of claim 20, wherein step of administering is selected from the group consisting of topical delivery, intravenous injection, and local injection.

22. The method of claim 20 further comprising detecting the acoustic wave and utilizing the detected acoustic wave to generate an image of the biological tissue.

23. A system comprising: a biological tissue; a bioconjugate disposed within the tissue, wherein the bioconjugate comprises a nanoparticle and a moiety capable of selectively coupling a molecular marker; and an imaging system comprising an ultrasonic sensor which emits an acoustic wave into the biological tissue and detects echoes of the acoustic wave.
